(19) United States
(12) Patent Application Publication
(10) Pub. No.: US 2026/0144737 A1
(43) Pub. Date: May 28, 2026

(54) Care and/or Make-Up Composition for Keratin Materials Comprising at Least One Volatile Solvent, a Particular Polyester and a Colorant

(71) Applicant: L'OREAL, Paris (FR)

(72) Inventors: NATHALIE GUILLIER, Chevilly Larue (FR); CAMILLE MOYNE, Chevilly Larue (FR); PASCAL ARNAUD, Chevilly Larue (FR)

(73) Assignee: L'OREAL, Paris (FR)

(21) Appl. No.: 19/121,864

(22) PCT Filed: Oct. 6, 2023
(86) PCT No.: PCT/EP2023/077756

§ 371 Date: Apr. 17, 2025

(30) Foreign Application Priority Data
Oct. 19, 2022 (FR) FR2210795

(51) Int. Cl. A61K 8/85; A61Q 1/06; A61Q 1/10

(52) U.S. Cl. CPC A61K 8/85; A61Q 1/06; A61Q 1/10; A61K 2800/432; A61K 2800/436; A61K 2800/49; A61K 2800/592

(57) ABSTRACT

Care and/or make-up composition for keratin materials comprising at least one volatile solvent, a particular polyester and a colorant The present invention relates to a liquid composition for caring for and/or making up keratin materials, comprising: A) at least one volatile solvent; B) at least one polyester which is the reaction product of the following components (i), (ii) and (iii): (i) at least one polyglycerol-3; (ii) at least one dimer acid; and (iii) at least one fatty monoacid having from 8 to 30 carbon atoms, the components (i), (ii) and (iii) reacted being in a molar ratio of 1 mole of polyglycerol-3, of 0.5 to 1 mole of dimer acid and of 0.1 to less than 2.0 moles of fatty monoacid; and C) optionally at least one non-volatile oil; the ratio by weight of the total amount of non-volatile oil(s) to the amount of polyester being less than 8.0; and D) at least one colorant. The invention also relates to a method for coating keratin materials, more particularly for making up and/or caring for keratin materials, such as the skin, characterized in that it comprises the application to keratin materials of a composition as defined above.

### TECHNICAL FIELD

The present invention is targeted at providing, for the field of the care and/or make-up of keratin materials, in particular of the skin, lips, eyelashes and eyebrows, a composition comprising a particular polyester.

Many cosmetic make-up compositions containing colorants, such as foundations, concealers, lipsticks, lip glosses or mascaras, have been developed for a longer wear property and non-transfer properties. This is achieved by the use of compositions which form a film after application. Such compositions generally contain volatile solvents which evaporate on contact with the skin or other keratin material, leaving behind a layer comprising waxes and/or film-forming polymers, pigments and fillers. However, these compositions tend to be sensorially uncomfortable for the consumer.

A poor wear property over time can be reflected in particular by a poor wear property over time of the colour and/or of the composition. This poor wear property can be characterized by a modification of the colour (change in colour, fading) generally owing to an interaction with sebum and/or moisture, such as sweat, secreted by the skin, in the case of a foundation, or to an interaction with saliva, in the case of lipsticks. This obliges the user to reapply the make-up very regularly, which may be time consuming.

It has already been proposed, in make-up compositions of the prior art, to use liquid or pasty polyesters to obtain wear properties.

Mention may in particular be made of the documents JP2002-128623, JP2002-128628, JP2002-128629 and EP 1 604 634, which describe polyesters of dilinoleic diacids and dilinoleyl diol dimers with the INCI name Dimer Dilinoleyl Dimer Dilinoleate, such as those sold by Nippon Fine Chemical under the trade names Lusplan DD-DA5® and DD-DA7®.

Mention may also be made of Patent EP 1 857 091 B1 describing polyesters obtained by reaction a) of diglycerol, b) of isostearic acid and c) of a dimer acid, such as dilinoleic diacid, in a molar ratio a)/b)/c) of 1/1.4 to 1.6/0.5 to 0.8, such as the polyester with the INCI name Polyglyceryl-2 Isostearate/Dimer Dilinoleate Copolymer sold under the trade name Hailucent Isda® by Kokyu Alcohol Kogyo.

To obtain hold properties in make-up compositions, there have also been provided, in the document FR29316739, polyesters obtained by condensation of dimer and/or trimer of unsaturated fatty acids and of diol; in particular, the polyester obtained by condensation of dimer and/or trimer of unsaturated fatty acid and of diol is a polyester of dilinoleic acid and of 1,4-butanediol, such as the polymer sold by Biosyntehsis under the name Viscoplast 14436H® (INCI name: Dilinoleic Acid/Butanediol Copolymer).

It is also known to use, in particular in the documents FR 2 931 069, JP2005-325079 and JP2006-28129, polyesters of hydroxylated fatty acid triglyceride and of a saturated fatty diacid for providing the wear property to make-up compositions. Mention may in particular be made, by way of examples of polyesters, of those with the INCI name Hydrogenated Castor Oil/Sebacic Acid Copolymer, such as the product sold under the name Crodabond CSA® by Croda, and also the hydrogenated castor oil dimer dilinoleate with the INCI name: Hydrogenated Castor Oil Dimer Dilinoleate, such as the product sold under the names Risocast-DA-L® and Risocast DA-HR by Kokyu Alcohol Kogyo.

Other pasty glycerol polyesters, in particular diglycerol polyesters, in particular Bis-Diglyceryl Polyacyladipate-2 sold under the trademark Softisan 649® by Sasol, have been proposed in make-up compositions, such as mascaras, in the document WO2017103235.

The Applicant Company has found, during its research studies, that the liquid make-up compositions comprising the polyesters mentioned above were not fully satisfactory in terms of wear property of make-up and of non-transfer for foundations, and in terms of resistance to moisture and to rubbing actions for mascaras. The Applicant Company has also observed that liquid products for making up the lips comprising the polyesters mentioned above led to rather unsatisfactory results for adhesion of the film and for make-up removal. These polyesters can result, on application, in dewetting or in a runny film between the two lips after the lips have been pressed against each other (blocking). In addition, the Applicant Company has observed that the films broke up into pieces on make-up removal.

The need remains to find new liquid formulae for caring for and/or making up keratin materials based on an appropriate polyester resulting in a good wear property of the deposit (rubbing actions, moisture) and in good non-transfer properties, without the disadvantages mentioned above.

During its research studies, the Applicant Company has unexpectedly discovered that these objectives could be achieved with a liquid composition for caring for and/or making up keratin materials, comprising in particular, in a physiologically acceptable medium:
A) at least one volatile solvent;
B) at least one polyester which is the reaction product of the following components (i), (ii) and (iii):
(i) at least one polyglycerol-3;
(ii) at least one dimer acid; and
(iii) at least one fatty monoacid having from 8 to 30 carbon atoms, the components (i), (ii) and (iii) reacted being in a molar ratio of 1 mole of polyglycerol-3, of 0.5 to 1 mole of dimer acid and of 0.1 to less than 2.0 moles of fatty monoacid; and
C) optionally at least one non-volatile oil; the ratio by weight of the total amount of non-volatile oil(s) to the amount of polyester being less than 8.0; and
D) at least one colorant.

This discovery forms the basis of the invention.

### Subject Matters of the Invention

Thus, according to one of its aspects, the present invention relates to a liquid composition for caring for and/or making up keratin materials, comprising in particular, in a physiologically acceptable medium:
A) at least one volatile solvent; and
B) at least one polyester which is the reaction product of the following components (i), (ii) and (iii):
(i) at least one polyglycerol-3;
(ii) at least one dimer acid; and
(iii) at least one fatty monoacid having from 8 to 30 carbon atoms, the components (i), (ii) and (iii) reacted being in a molar ratio of 1 mole of polyglycerol-3, of 0.5 to 1 mole of dimer acid and of 0.1 to less than 2.0 moles of fatty acids; and
C) optionally at least one non-volatile oil; the ratio by weight of the total amount of non-volatile oil(s) to the amount of polyester being less than 8.0; and
D) at least one colorant.

The invention also relates to a method for coating keratin materials, more particularly for making up and/or caring for keratin materials, such as the skin, the lips, the eyelashes and the eyebrows, characterized in that it comprises at least the application to keratin materials of a composition as defined above.

### DEFINITIONS

In the context of the present invention, the term “keratin material” is understood in particular to mean the skin (body, face, outline of the eyes), the lips, the eyelashes and the eyebrows.

The term “physiologically acceptable” is understood to mean compatible with the skin and/or its superficial body growths, which exhibits a pleasant colour, odour and feel and which does not cause unacceptable discomfort (stinging, tautness) liable to dissuade the consumer from using this composition.

The term “liquid composition” is understood to mean any composition which exhibits at least one of the following characteristics:
i) flows under its own weight at ambient temperature (20-25° C.) and at atmospheric pressure (760 mmHg or 1.013×10⁵ Pa);
ii) is not solid at ambient temperature and at atmospheric pressure and of which it is possible to measure a viscosity or its consistency characterized by its hardness;
iii) does not exhibit any particular shape such as that which can be obtained by hot casting in a mould or container of a given shape.

Such compositions can thus be found in particular in fluid, creamy, pasty or gel form.

The term “polyester” is understood to mean any polymer obtained by a condensation reaction of polycarboxylic acids with alcohols or glycols. Its macromolecular backbone contains the repetition of its ester function. The ester function denotes a characteristic group formed of an atom bonded simultaneously to an oxygen atom by a double bond and to an alkoxy group. When the bonded atom is a carbon atom, it is called a carboxylic ester, the general formula of which is R—COO—R′.

The term “polyglycerol-3” is understood to mean triglycerol alone or a mixture of polyglycerols comprising at least triglycerol, and preferably triglycerol is predominant in said mixture.

### Protocol for the Measurement of the Viscosity:

The viscosity measurement is generally carried out at 25° C., using a Rheomat RM 180 viscometer equipped with a No. 2, 3 or 4 spindle, the measurement being carried out after 10 minutes of rotation of the spindle within the composition (time on completion of which stabilization of the viscosity and of the rotational speed of the spindle are observed), at a speed of 200 revolutions/min (rpm).

According to one embodiment, the composition according to the invention can exhibit, at 25° C., a viscosity of between 0.1 and 25 Pa·s, preferably of between 0.2 and 20 Pa·s.

Preferably, the viscosity at 25° C. of a composition according to the invention may be between 0.2 and 10 Pa·s.

In particular, the viscosity at 25° C. of a composition according to the invention can be of between 0.1 Pa·s (spindle 2) and 25 Pa·s (spindle 4), preferably between 0.2 Pa·s (spindle 2) and 20 Pa·s (spindle 4) and better still between 0.2 Pa·s (spindle 2) and 10 Pa·s (spindle 4).

### Protocol for the Measurement of the Consistency

According to a particular form, the composition according to the invention exhibits, at 25° C., a consistency characterized by a hardness of less than or equal to 250 g preferentially, a hardness ranging from 20 to 150 g, more preferentially still from 25 to 100 g.

The consistency can be measured according to the following protocol:

The measurement device is a TA-XT-Plus® sold by Staples Micro System, equipped with a cell for measuring a force of 5 kilograms and with a cylindrical spindle 12.7 mm (½ inch) in diameter made of Delrin. The composition is thermostatically controlled at 20° C. It is then placed in excess in a container with a diameter of 60 mm and a depth of 22 mm using a metal spatula. The product is spread out so as to avoid any air pockets but without pummelling it, so as not to destructure it. The container is subsequently leveled off using a spatula so as to have a surface which is as uniform as possible. The container is subsequently covered with a watch glass so as to limit the evaporation of solvents present in the formula for about 10 minutes. The options chosen for this measurement method are as follows:

### Test Mode: Compression Measurement

Trigger force: 2.0 g
Pre-speed: 0.5 mm/sec
Test speed: 0.5 mm/sec
Temperature: 20° C.±1° C.
Penetration distance: 5 mm

Three successive measurements are carried out at points at least 12 mm apart, at least 10 mm from the edge of the container. The container is held in place during the measurement. The value retained is the mean of the maxima obtained at each measurement.

### Volatile Solvents

The composition in accordance with the present invention comprises at least one volatile solvent.

In the context of the invention, the term “volatile solvent” is understood to mean a compound which is liquid at ambient temperature (20° C.) and at atmospheric pressure (760 mmHg) exhibiting a vapour pressure at 20° C. of greater than 0.1 mmHg and preferably of between 0.1 and 300 mmHg, more preferentially still between 0.5 and 200 mmHg.

Mention may be made, among the volatile solvents, of:
water;
monoalcohols comprising from 2 to 6 carbon atoms;
esters of liquid acids and of alcohols, such as methyl acetate, n-butyl acetate, ethyl acetate, propyl acetate, isopentyl acetate or ethyl 3-ethoxypropionate;
ketones which are liquid at ambient temperature and volatile, such as methyl ethyl ketone, methyl isobutyl ketone, diisobutyl ketone, isophorone, cyclohexanone or acetone;
volatile polyols, such as propylene glycol;
volatile ethers, such as dimethoxymethane, diethoxyethane or diethyl ether;
volatile glycol ethers such as 2-butoxyethanol, butyl diglycol, diethylene glycol monomethyl ether, propylene glycol n-butyl ether or propylene glycol monomethyl ether acetate;
volatile oils.

According to a particular form of the invention, the volatile solvent is chosen from:
water:
monoalcohols comprising from 2 to 6 carbon atoms;
volatile oils;
their mixtures.

### Water

A water suitable for the invention can be a demineralized water, a floral water, such as cornflower water, and/or a mineral water, such as Vittel water, Lucas water or La Roche-Posay water, and/or a thermal water.

C₂-C₆ Monoalcohol

The monoalcohol(s) in accordance with the invention comprise, preferably, from 2 to 6 carbon atoms and in particular from 2 to 4 carbon atoms and their mixtures.

The monoalcohol(s) can be represented, for example, by the formula RaOH, in which Ra represents a linear or branched alkyl group comprising from 2 to 6 carbon atoms.

Mention may be made, by way of monoalcohol, of ethanol, isopropanol, propanol or butanol, and more particularly of ethanol.

According to an advantageous embodiment, the amount of monoalcohol(s) varies from 0% to 60% by weight, preferably from 2% to 50% by weight and more preferentially still from 3% to 40% by weight, with respect to the total weight of said composition.

### Volatile Oil

The term “oil” is understood to mean any fatty substance in the liquid form at ambient temperature (25° C.) and at atmospheric pressure (760 mmHg or 10⁵ Pa).

The volatile oils in accordance with the invention can be chosen from the group constituted of hydrocarbon oils, silicone oils and their mixtures.

Within the meaning of the present invention, the term “silicone oil” denotes an oil comprising at least one Si—O group, and more particularly an organopolysiloxane.

The term “hydrocarbon oil” is understood to mean an oil containing predominantly hydrogen and carbon atoms and optionally one or more functions chosen from hydroxyl, ester, ether and carboxyl functions.

Within the meaning of the invention, the term “volatile oil” is understood to mean any oil capable of evaporating on contact with the skin in less than one hour, at ambient temperature and atmospheric pressure. The volatile oil is a volatile cosmetic compound, liquid at ambient temperature, having in particular a non-zero vapour pressure, at ambient temperature and atmospheric pressure, in particular having a vapour pressure ranging from 2.66 Pa to 40 000 Pa, especially ranging from 2.66 Pa to 13 000 Pa and more particularly ranging from 2.66 Pa to 1300 Pa.

Mention may be made, as example of volatile silicone oil which can be used in the invention, of volatile silicone oils, such as volatile linear or cyclic silicone oils, in particular those having a viscosity of 2 to 8 centistokes (2×10⁻⁶ to 8×10⁻⁶ m²/s), and containing in particular from 2 to 7 silicon atoms, these silicones optionally comprising alkyl or alkoxy groups containing from 1 to 10 carbon atoms. Mention may in particular be made, as volatile silicone oils which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane; and their mixtures.

The volatile hydrocarbon oils which can be used in the compositions according to the invention can be chosen from branched C₈-C₁₆ alkanes.

Mention may in particular be made, as C₈-C₁₆ isoalkanes of petroleum origin (also known as isoparaffins), such as isododecane (also known as 2,2,4,4,6-pentamethylheptane), isodecane, isohexadecane and, for example, the oils sold under the Isopar® or Permethyl® trade names.

Mention may also be made of branched C₈-C₁₆ esters, such as isohexyl neopentanoate. Other volatile hydrocarbon oils, such as petroleum distillates, in particular those sold under the name Shell Solt® by Shell, can also be used.

The volatile hydrocarbon oils which can be used in the compositions according to the invention can be chosen from volatile linear alkanes comprising from 6 to 14 carbon atoms.

Mention may be made, by way of examples of linear alkanes suitable for the invention, of the alkanes described in the patent applications WO 2007/068371 and WO 2008/155059 of Cognis (mixtures of different alkanes differing by at least one carbon). These alkanes are obtained from fatty alcohols, themselves obtained from coconut oil or palm oil.

Mention may be made, by way of examples of linear C₆-C₁₄ alkanes suitable for the invention, of n-hexane (C₆), n-heptane (C₇), n-octane (C₈), n-nonane (C₉), n-decane (C₁₀), n-undecane (C₁₁), n-dodecane (C₁₂), n-tridecane (C₁₃), n-tetradecane (C₁₄) and their mixtures.

Mention may in particular be made of n-dodecane (C₁₂) and n-tetradecane (C₁₄), sold by Sasol respectively under the references Parafol 12-97® and Parafol 14-97®, and also their mixtures.

According to another embodiment, a mixture of n-dodecane and n-tetradecane is used. It is in particular possible to use the dodecane/tetradecane mixture in the 85/15 ratio by weight sold by Biosyntehsis under the reference Vegelight 1214®.

According to yet another embodiment, use is made of a mixture of volatile linear C₉-C₁₂ alkanes with the INCI name: C₉-12 Alkane, such as the product sold by Biosyntehsis under the reference Vegelight Silk®.

According to yet another embodiment, use is made of a mixture of n-undecane (C₁₁) and of n-tridecane (C₁₃), like those obtained in Examples 1 and 2 of Application WO 2008/155059 from Cognis and like that sold under the trade name Cetiol Ultimate® by BASF.

According to a particularly preferred embodiment, the volatile hydrocarbon oil is chosen from branched C₈-C₁₆ alkanes, and more particularly isododecane, the mixture of volatile linear C₉-C₁₂ alkanes and the mixture of n-undecane (C₁₁) and of n-tridecane (C₁₃).

The volatile oil(s) are preferably present in the composition of the invention at contents of less than 60.0% by weight, preferably of less than 50.0% by weight and more preferentially still of less than 40.0% by weight, with respect to the total weight of said composition.

According to a preferential form of the invention, the volatile solvent(s) are present in the composition of the invention at contents ranging from 5% to 75% by weight, preferably from 10% to 70% by weight and more preferentially still from 20% to 65% by weight, with respect to the total weight of said composition.

Preferably, the ratio by weight of the total amount of volatile solvent(s) to the amount of polyester is greater than 1.0.

Polyglycerol-3/Dimer Acid/Fatty C₈-C₃₀ Monoacid Polyester

The composition in accordance with the invention comprises at least one polyester which is the reaction product of the following components (i), (ii) and (iii):
(i) at least one polyglycerol-3;
(ii) at least one dimer acid; and
(iii) at least one fatty monoacid having from 8 to 30 carbon atoms, the components (i), (ii) and (iii) reacted being in a molar ratio of 1 mole of polyglycerol-3, of 0.5 to 1 mole of dimer acid and of 0.1 to less than 2.0 moles of fatty acids.

The polyesters of the invention, and also their synthesis, are described in Patent Applications US 2021/10259945, US 2021/10259946 and US 2021/10259930.

According to a preferred embodiment, the amount, as active material, of polyester varies from 1% to 50% by weight, more preferentially from 1.5% to 30% by weight and more preferentially still from 2% to 20% by weight, with respect to the total weight of the composition.

According to a preferred embodiment, the polyester is a substantially or completely non-sequential reaction product.

The term “substantially non-sequential reaction product” is understood to mean the product obtained by a substantially non-sequential reaction of the reactive components (i)-(iii).

The term “completely non-sequential reaction of the reactive components (i)-(iii)” is understood to mean that the total content of each of the reactants (i)-(iii) to be made to react is added to the reaction vessel before starting the reaction.

In one embodiment of the present invention, the total content of each of the reactants (i)-(iii) to be made to react is added to the reaction vessel before starting the reaction, that is to say that the reaction is completely non-sequential, and the polymer is a completely non-sequential reaction product of the components (i)-(iii). In other embodiments, 70-100%, or 75-100%, or 80-100%, or 85-100%, or 90-100%, or 95-100%, or 97-100% of each of the reactants (i)-(iii) is added to the reaction vessel before starting the reaction.

In one embodiment, the polyester is prepared by a one-stage process which involves the introduction of all the reactants into a reaction vessel and subsequently the induction of an entirely random addition of the dimer acid and of the isostearic acid to the polyglycerol-3.

Triglycerol has the formula H—[—OGly]₃-OH in which Gly designates a glycerol residue after removal of two hydroxyl groups.

A polyglycerol-3 according to the invention in the form of a mixture of polyglycerols containing at least triglycerol comprises polyglycerols which can be any product of oligocondensation of glycerol. They preferably correspond to the formula (I):

[Chem 1]

H[—O-Gly-]ₙ—OH (I)

in which each Gly is independently the residue of a glycerol molecule after removal of two hydroxyl groups; and n is a mean from 2 to 10.

Generally, the majority of the Gly groups are of the formula: —CH₂—CHOH—CH₂—, although the residues comprising etherification at secondary or even tertiary hydroxyl groups are regarded as being within the scope of “Gly” and, consequently, may also be present.

Examples of polyglycerol-3 in the form of a mixture comprise diglycerol, triglycerol, tetraglycerol, pentaglycerol, hexaglycerol, heptaglycerol, octaglycerol, nonaglycerol, decaglycerol and mixtures of these. In particular, preferential polyglycerols are those of formula (I) in which n in particular has a value from 2 to 7, more particularly from 2 to 5 and in particular 2, 3 or 4, or mixtures of polyglycerols in these ranges.

Particularly appropriate examples of polyglycerol-3 comprise a mixture of polyglycerols having the following distribution, in which all the percentages by weight are based with respect to the total weight of the polyglycerol-3 in the form of a mixture:
glycerol: from 0% to 30% by weight, preferably from 0% to 20% by weight, in a preferred way from 0% to 15% by weight;
diglycerol: from 10% to 40% by weight, preferably from 15% to 35% by weight, in a preferred way from 20% to 32% by weight;
triglycerol: from 10% to 65% by weight, preferably from 15% to 60% by weight, in a preferred way from 18% to 55% by weight;
tetraglycerol: from 2% to 25% by weight, preferably from 5% to 20% by weight, in a preferred way from 8% to 20% by weight;
pentaglycerol: from 0% to 15% by weight, preferably from 0% to 10% by weight, in a preferred way from 0% to 5% by weight;
hexaglycerol: from 0% to 15% by weight, preferably from 0% to 10% by weight, in a preferred way from 0% to 5% by weight;
heptaglycerol: from 0% to 10% by weight, preferably from 0% to 5% by weight, in a preferred way from 0% to 3% by weight;
octaglycerol: from 0% to 10% by weight, preferably from 0% to 5% by weight, in a preferred way from 0% to 3% by weight;
nonaglycerol: from 0% to 5% by weight, preferably from 0% to 3% by weight, in a preferred way from 0% to 2% by weight;
decaglycerol: from 0% to 5% by weight, preferably from 0% to 3% by weight, in a preferred way from 0% to 2% by weight.

In one embodiment, a polyglycerol-3 in the form of a mixture comprises the following distribution of polyglycerols:
Glycerol: from 0% to 30% by weight;
Diglycerol: from 15% to 40% by weight;
Triglycerol: from 10% to 55% by weight;
Tetraglycerol: from 2% to 25% by weight;
Pentaglycerol and higher components: from 0% to 15% by weight, with respect to the total weight of the polyglycerol-3 in the form of a mixture.

In one embodiment, a polyglycerol-3 in the form of a mixture is composed of at least 40% by weight, or of at least 45% by weight, or of at least 50% by weight, of a combination of diglycerol and of triglycerol, with respect to the total weight of the polyglycerol-3 in the form of a mixture.

In one embodiment, a polyglycerol-3 is composed of at least 20% by weight, or of at least 25% by weight, of diglycerol; at least 15% by weight, or at least 18% by weight, of triglycerol; at least 10% by weight, or at least 12% by weight, of tetraglycerol; in which all the percentages by weight are with respect to the total weight of the polyglycerol-3 in the form of a mixture.

A particularly preferred polyglycerol-3 comprises at least 25% by weight of diglycerol, at least 45% by weight of triglycerol and at least 10% by weight of tetraglycerol, with respect to the total weight of the polyglycerol-3 in the form of a mixture.

Analysis of such a polyglycerol-3 composition can be carried out in order to determine its median or “mean” polyglycerol number. The examples of polyglycerols above with narrow and broad distributions can also be denoted as polyglycerol-3 because it is a matter of the integer closest to the mean and/or median.

### Dimer Acid

The dimer acid can be any dicarboxylic acid having at least 4 carbon atoms. They can be linear or branched, such as, for example, the dimers prepared from malonic acid, succinic acid, fumaric acid, dimethylglutaric acid or trimethyladipic acid, and from their anhydrides.

Dimer fatty acids are particularly useful. As is known, these are mixtures of acyclic and cyclic dicarboxylic acids which are obtained by a catalysed dimerization reaction of unsaturated fatty acids having from 12 to 22 carbon atoms.

For the preparation and the use of dimer acids and their physical and chemical properties, reference will be made to the publication “The Dimer Acids: The Chemical and Physical Properties, Reactions and Applications”, Ed. E. C. Leonard; Humko Sheffield Chemical, 1975, Memphis, Tenn.

The dicarboxylic acids can also contain, to a lesser extent, tri- and polyfunctional carboxylic acids. The functionality of the mixture must not exceed a mean molar value of 2.4.

Preferred dimer acids are typically derived from triglycerides rich in C₁₈ ester groups, which can be hydrolysed to produce unsaturated fatty C₁₈ monoacids. The starting materials can be derived from tallow oil and rapeseed oil but other natural sources, such as flax seeds, soybean, pumpkin and walnut, can be used. The target monoacids used in the reaction are rich in forms of oleic and linoleic acids which are described in the list of fatty acids which is contained below. Dimerization results mainly in the dimerization of unsaturated fatty acids, but trimers are also formed. After reaction, the product can be stored in the form of a mixture of reaction products or it can be further distilled or otherwise separated into molecular weight fractions. In one embodiment, the dimerization reaction produces a predominance (at least 60% by weight, more preferably at least 75% by weight) of dimer acid (C₃₆ diacid) but also produces C₅₄ trimer acids (less than 30% by weight, more preferably less than 25% by weight).

In one case, a standard dimer acid commercially available from Croda, Pripol 1025®, which contains 72% by weight of dimer and 19% by weight of trimer acid, is used.

In another case, a hydrogenated standard dimer acid from Oleon, Radiacid 0960®, which contains 87% by weight of dimer and 10% by weight of trimer acid, is used. In both cases, the polymer as described is characterized by a higher molecular weight, a more hydrophobic nature and a higher viscosity than those which can be provided by pure diacids of lower molecular weight. The presence of trimer acid further improves the molecular weight and the performance qualities of these polymers.

In one embodiment, the copolymer of the present invention is prepared from at least one hydrogenated dimer acid.

In another embodiment, the polymer is prepared from a hydrogenated dimer acid comprising hydrogenated dimerized C₁₈ fatty acids, which hydrogenated dimer acid is obtained by dimerization of unsaturated C₁₈ fatty acids and subsequent hydrogenation.

In one embodiment, the hydrogenated dimer acid contains a content of trimer acid ranging from approximately 5% to 25% by weight, based on the total weight of hydrogenated dimer acid.

In another embodiment, the hydrogenated dimer acid contains a predominance (at least 60% by weight, more preferentially at least 75% by weight, but at most 95% by weight, or better still at most 90% by weight, or even better still at most 85% by weight of hydrogenated dimer acid (C₃₆ diacid) and also contains hydrogenated C₅₄ trimer acids (less than 30% by weight, more preferably less than 25% by weight, but more than 5% by weight, more preferably more than 10% by weight).

Fatty C₈-C₃₀ Monoacid

Fatty C₈-C₃₀ monoacids can include natural or refined fatty acids, such as hydrolysed rapeseed oil, sunflower oils, and the like, but these contain both lower and higher MW chains. Useful fatty monoacids can be linear, branched, saturated, unsaturated and aromatic materials with an acidity provided by carboxylic acid fractions.

Acids suitable for the invention comprise caprylic acid (C₈), pelargonic acid (C₉), capric acid (C₁₀), undecylic acid (C₁₁), lauric acid (C₁₂), tridecylic acid (C₁₃), myristic acid (C₁₄), pentadecylic acid (C₁₅), palmitic acid (C₁₆), margaric acid (C₁₇), stearic acid (C₁₈), isostearic acid (C₁₈), nonadecylic acid (C₁₉), arachidic acid (C₂₀), behenic acid (C₂₂) and lignoceric acid (C₂₄).

The comparison of stearic acid and isostearic acid shows that the branching leads to an elevated melting point and results in a low viscosity at ambient temperature for isostearic acid, compared to a solid material for stearic acid. This lower viscosity can be useful in the handling of starting materials and also to make it possible for the esters manufactured with this acid to retain their liquid properties. Branched-chain fatty acids often contain a single methyl branch along the linear carbon chain and are produced in nature by microbial action. Isostearic acid is available as a reaction by-product in the creation of the dimer acid described above.

Another way to obtain a liquid product consists in using unsaturated, linear and branched, fatty monoacids. These unsaturated acids can include palmitoleic acid (C16:1), vaccenic acid (C18:1), oleic acid (C18:1), elaidic acid (C18:1), linoleic acid (C18:2), linolelaidic acid (C18:2), α-linolenic acid (C18:3), γ-linolenic acid (C18:3), stearidonic acid (C18:4), paullinic acid (C20:1), gondoic acid (C20:1), dihomo-γ-linolenic acid (C20:3), mead acid (C20:3), arachidonic acid (C20:4), eicosapentaenoic acid (C20:5), erucic acid (C22:1), docosatetraenoic acid (C22:4), cervonic acid (C22:6) and nervonic acid (C24:1). As is well known to a person skilled in the art, the designation means that the length of the carbon chain is X carbon atoms and that there are Y double bonds in the chain.

In one embodiment, isostearic acid will be preferred.

In a particularly preferred embodiment, the polyester of the invention is a substantially or completely non-sequential reaction product of the following components:
(i) at least one polyglycerol-3 in the form of a mixture comprising at least 25% by weight of diglycerol, at least 45% by weight of triglycerol and at least 10% by weight of tetraglycerol, with respect to the total weight of polyglycerol-3 in the form of a mixture;
(ii) at least one hydrogenated dimer acid containing at least 60% by weight of hydrogenated C36 diacid and from 5% to 25% by weight of hydrogenated C54 triacid, in each case with respect to the total weight of hydrogenated acid; and iii) isostearic acid.

In one embodiment, the polyester is prepared by a one-stage process which involves the introduction of all the reactants into a reaction vessel and subsequently the induction of an entirely random addition of the dimer acid and of the isostearic acid to the polyglycerol-3.

In one embodiment, it is preferable to have a total degree of esterification of the available polyglycerol hydroxyl fragments (total esterification) of 24% to 74% and a degree of esterification of the available polyglycerol hydroxyl fragments by a dimer acid alone (esterification with a dimer acid) of from 20% to 40%. More important still, the degree of esterification by end-cap units (esterification with a monoacid) is also defined in this description and it is important to maintain the esterification with a monoacid from 4% to 40%.

It is preferable to have a total esterification of 28% to 57% with an esterification with a dimer acid of 20% to 30% and an esterification with a monoacid between 8% and 27%.

It is even more preferable to have a total esterification of 33% to 48% with an esterification with a dimer acid of 20% to 28% and an esterification with a monoacid between 13% and 20%.

It is even more preferable to have a total esterification of 24% to 74% with an esterification with a hydrogenated dimer acid of 20% to 40% and an esterification with a monoacid between 4% and 40%.

It is even more preferable to have a total esterification of 28% to 57% with an esterification with a hydrogenated dimer acid of 20% to 30% and an esterification with a monoacid between 8% and 27%.

It is also even more preferable to have a total esterification of approximately 40% with an esterification with a hydrogenated dimer acid of approximately 20% and an esterification with a monoacid of approximately 20%.

It is also even more preferable to have a total esterification of approximately 40% with an esterification with a hydrogenated dimer acid of approximately 27% and an esterification with a monoacid of approximately 13%.

In one embodiment, the reacted components are in a molar ratio of 1 mole of polyglycerol-3, 0.5 to 1 mole of dimer acid and 0.2 to 1.7 mole of fatty acid.

In another embodiment, the reacted components are in a molar ratio of 1 mole of polyglycerol-3, 0.5 to 0.75 mole of dimer acid and 0.4 to 1.35 mole of isostearic acid.

In another embodiment, the reacted components are in a molar ratio of 1 mole of polyglycerol-3, 0.5 to 0.7 mole of dimer acid and 0.65 to 1 mole of isostearic acid.

In another embodiment, the reacted components are in a molar ratio of 1 mole of polyglycerol-3, 0.5 to 1 mole of hydrogenated dimer acid and 0.2 to 1.7 mole of isostearic acid.

In another embodiment, the reacted components are in a molar ratio of 1 mole of polyglycerol-3, 0.5 to 0.75 mole of hydrogenated dimer acid and 0.4 to 1.35 mole of isostearic acid.

In another embodiment, the reacted components are in a molar ratio of 1 mole of polyglycerol-3, 0.5 to 0.7 mole of hydrogenated dimer acid and 0.65 to 1 mole of isostearic acid.

In another embodiment, the reacted components are in a molar ratio of 1 mole of polyglycerol-3, 0.5 to 1 mole of hydrogenated dimer acid and 0.2 to 1.7 mole of isostearic acid.

In another embodiment, the reacted components are in a molar ratio of 1 mole of polyglycerol-3, 0.5 to 0.75 mole of hydrogenated dimer acid and 0.4 to 1.35 mole of isostearic acid.

In another embodiment, the reacted components are in a molar ratio of 1 mole of polyglycerol-3, 0.5 to 0.7 mole of hydrogenated dimer acid and 0.65 to 1 mole of isostearic acid.

In another embodiment, the reacted components are in a molar ratio of 1 mole of polyglycerol-3, 0.67 mole of hydrogenated C₃₆ dimer acid and 0.67 mole of isostearic acid.

In a particularly preferred embodiment, the reacted components are in a molar ratio of 1 mole of polyglycerol-3, 0.5 mole of hydrogenated C₃₆ dimer acid and 1 mole of isostearic acid.

By adjusting the molar ratio of the termination of the fatty acids and by balancing the amount of polyglycerol-3 and of dimer acid, it is also possible to control the degree of dimer acid-polyglycerol extension and termination so that crosslinking, for example, via the trimer acid, results in much higher viscosities.

The target viscosity of the pure polymer must be >50 000 mPa·s and less than 5 000 000 mPa·s at 25° C.

In a preferred embodiment, the target viscosity is >75 000 mPa·s and <2 500 000 mPa·s at 25° C.

In another preferred embodiment, the target viscosity is >100 000 mPa·s and <2 000 000 mPa·s at 25° C.

In a most preferred embodiment, the target viscosity is >1 000 000 mPa·s and <2 000 000 mPa·s at 25° C.

The viscosity is measured using an MCR3O2® rheometer from Anton Paar Inc. Rough or smooth twin flat plates 50 mm in diameter were used, covered with a polymer sample, adjusted to a gap of 0.5 to 1 mm, and temperature and shear rate scans were carried out. The polyesters of the invention exhibit a Newtonian behaviour and thus have a constant viscosity over a wide range of shear rates. In addition, the polymers of this description have demonstrated a reduced viscosity with temperature. Thus, the viscosity measurements are reported at a precisely controlled temperature and generally in the form of a shear rate of 1. The values are reported in mPa·s.

The polyesters of the invention are characterized by weight-average molecular weights >2500 Da and <1 000 000 Da, measured by GC using linear polystyrene standards.

The GC column used for these tests constituted of: Phenolgel, 300× 4.6 mm; a continuous tetrahydrofuran (THF) phase was used and injected at 0.35 ml/min, column oven maintained at 40° C.; a 50 μl injection and a Wyatt Ri refractive index detector. The calibration standards used were strictly linear polystyrene intended to be monodispersed. The narrow range polystyrene GC calibrating standards were prepared as a mobile phase and had maximum molecular weights of 1 290 000 Da, 560 000 Da, 65 500 Da, 28 500 Da, 10 100 Da, 1680 Da, 580 Da and 208 Da. Using standard methodologies, the weight- and number-average molecular weight is automatically calculated by standard GC software.

In a preferred embodiment, the polyesters described have a weight-average molecular weight >4000 Da and <250 000 Da, measured by GC using linear polystyrene standards. In a most preferred embodiment, the polymers described have a weight-average molecular weight >5000 Da and <150 000 Da, measured by GC using linear polystyrene standards.

In yet another embodiment, the polyester of the invention exhibits a combination of weight-average molecular weight >5000 Da and <150 000 Da, measured by GC using linear polystyrene standards, and of viscosity at 25° C.>100 000 mPa·s and <2 000 000 mPa·s.

In a preferred embodiment, the polyester of the invention is a substantially or completely non-sequential reaction product of the following components:
(i) at least one polyglycerol-3 comprising at least 25% by weight of diglycerol, at least 45% by weight of triglycerol and at least 10% by weight of tetraglycerol, in each case with respect to the total weight of polyglycerol-3 in the form of a mixture;
(ii) at least one hydrogenated dimer acid containing at least 60% by weight of hydrogenated C₃₆ diacid and from 5% to 25% by weight of hydrogenated C₅₄ triacid, in each case with respect to the total weight of hydrogenated acid; and
(iii) isostearic acid; in which the polymer exhibits a combination of weight-average molecular weight >5000 Da and <15 000 Da, measured with GC using linear polystyrene standards, and of viscosity of the pure polymer>100 000 mPa·s and <2 000 000 mPa·s at 25° C.; and in which the copolymer is also characterized by a complete esterification of approximately 40%, an esterification with a hydrogenated dimer acid of approximately 27% and an esterification with a monoacid of approximately 13%.

In practice, given that the starting ingredients contain a range of polyglycerol units and a range of dimer and trimer acid contents, the above numbers can be adjusted using the actual (and non-theoretical) hydroxyl fractions and carboxylic acid fractions, such as are determined by methods such as mass spectrometry, NMR and liquid chromatography. The above esterification ranges are based on the ideal structure of the polyglycerol-3 and of the C₃₆ dimer acid. The actual ranges can thus be slightly different from the values indicated above and can be calculated based on these analytical values.

It is more practical to define the extent of the polymerization by the final acid number. The initial acid values, in the light of the distribution of the polyglycerol, monoacid and polyacid fractions present, can be reliably calculated using the actual acid value determined by the starting ingredient used.

For an example, the initial total acid number (“AV”, which is commonly defined in mg of KOH/g of total reactant), is 135 AV. This includes 68 AV for the dimer acid and 67 AV for the isostearic acid for a preferred embodiment containing 1 mole of polyglycerol-3, 0.5 mole of hydrogenated C₃₆ dimer acid and 1 mole of isostearic acid. All the preferred ratio embodiments described above have a corresponding initial AV which can be calculated. When, during the polymerization reaction, the AV units are reduced, this ratio gives the percentage of conversion of the reaction from the total initial reactive acid fractions to the final residual acid fractions.

Thus, the completion rate of the reaction is defined by

[Math 1]

(1−final AV)/initial AV.

In one embodiment, the polyesters of the invention have final acid numbers from 0.1 to <25 mg of KOH/g of polymer.

In a preferred embodiment, the polyesters of the invention have final acid numbers from 0.1 to <10 mg of KOH/g of polymer.

In a most preferred embodiment, the polyesters of the invention have final acid numbers from 0.1 to <5 mg of KOH/g of polymer.

As the completion rate of the reaction is defined by the equation 1-final AV/initial AV, the completion rate of the reaction of such mixtures to give final polymer is >80%.

In a preferred embodiment, the completion rate of the reaction of such mixtures to give final polymer is >90%.

In a most preferred embodiment, the completion rate of the reaction of such mixtures to give final polymer is >95%.

In a preferred embodiment, the polyester of the invention is a reaction product of a polyglycerol-3, of a hydrogenated C₃₆ dimer acid and of isostearic acid in a molar ratio of 1/0.5/1, as described in Example 10 (copolymer) of the document US 2021/0259945.

### Non-Volatile Oils

According to a preferential form, the composition in accordance with the invention comprises at least one non-volatile oil; the ratio by weight of the total amount of non-volatile oil(s) to the amount of polyester is less than 8.0, and more preferentially varies from 0.5 to 5.

The term “non-volatile oil” is understood to mean an oil remaining on the skin or the keratin fibre at ambient temperature and atmospheric pressure for at least several hours and having in particular a vapour pressure of less than 2.66 Pa, preferably of less than 0.13 Pa. By way of example, the vapour pressure can be measured according to the static method or by the effusion method by isothermal thermogravimetry as a function of the vapour pressure (Standard OECD 104).

The non-volatile oils in accordance with the invention can be chosen from the group constituted of hydrocarbon oils, silicone oils and their mixtures.

Mention may be made, as examples of non-volatile oils which can be used in the invention, of:
hydrocarbon oils of vegetable origin, such as triglycerides of fatty acids containing from 4 to 24 carbon atoms, such as, for example, caprylic/capric triglycerides, such as those sold by Stearineries Dubois or those sold under the names Miglyol 810®, 812® and 818® by Dynamit Nobel; triglycerides of branched C₁₈-C₃₆ fatty acids and of glycerol, such as that sold under the name DUB TGI 24® by Stéarineries Dubois (INCI name: C₁₈-36 Acid Triglyceride);
linear or branched hydrocarbons, of mineral or synthetic origin, such as liquid paraffins and their derivatives, liquid petrolatum, polydecenes, polybutenes, hydrogenated polyisobutene, such as Parleam, or squalane;
synthetic ethers containing from 10 to 40 carbon atoms, such as dicaprylyl ether;
synthetic esters, in particular of fatty acids of formula R1COOR2, such as isononyl isononanoate, isopropyl myristate, isopropyl palmitate, C₁₂-C₁₅ alkyl benzoate, triheptanoin, hexyl laurate, isoamyl laurate, diisopropyl adipate, 2-ethylhexyl palmitate, 2-octyldodecyl stearate, 2-octyldodecyl erucate, isostearyl isostearate, heptyl undecylenate, diisostearyl malate and tridecyl trimellitate:
   fatty alcohols which are liquid at ambient temperature, comprising a branched and/or unsaturated carbon chain comprising from 12 to 26 carbon atoms, such as octyldodecanol, isostearyl alcohol, 2-butyloctanol, 2-hexyldecanol, 2-undecylpentadecanol or oleyl alcohol;
   higher fatty acids, such as oleic acid, linoleic acid or linolenic acid;
   carbonates, such as dicaprylyl carbonate;
   acetates;
   citrates;
   polydimethylsiloxanes (INCI name: Dimethicone) (in particular with a viscosity of 50 to 500 cSt (10⁻⁶ m²/s), in particular 350 cSt (10⁻⁶ m²/s), such as the commercial products sold under the names Belsil DM 350® from Wacker, and Xiameter PMX-200 Silicone Fluid® 350 CS from Dow Corning, and more particularly polydimethylsiloxanes (INCI name: Dimethicone) with a viscosity of 50 to 150 cSt (10⁻⁶ m²/s), in particular 100 cSt (10⁻⁶ m²/s), such as the commercial products sold under the names Belsil DM 100® from Wacker, and Xiameter PMX-200 Silicone Fluid 100 CS® from Dow Corning;
   phenylated silicones, such as phenyl trimethicones, phenyl dimethicones, phenyl(trimethylsiloxy) diphenylsiloxanes, diphenyl dimethicones, diphenyl(methyldiphenyl)trisiloxanes and (2-phenylethyl)trimethylsiloxysilicates; and
   their mixtures.

According to a preferred embodiment, the non-volatile oil will be chosen from non-volatile hydrocarbon oils and more preferentially chosen from triglycerides of fatty acids containing from 4 to 24 carbon atoms, and more particularly a triglyceride of caprylic/capric acids (INCI name: Caprylic/Capric Triglyceride).

According to a particularly preferred form of the invention, the composition comprises:
A) at least one volatile solvent as defined above; and
B) at least one oily solution comprising:
a) at least one polyester which is the reaction product of the following components (i), (ii) and (iii):
(i) at least one polyglycerol-3;
(ii) at least one dimer acid; and
(iii) at least one fatty monoacid having from 8 to 30 carbon atoms, the components (i), (ii) and (iii) reacted being in a molar ratio of 1 mole of polyglycerol, of 0.5 to 1 mole of dimer acid and of 0.1 to less than 2.0 moles of fatty acids; and
b) at least one non-volatile oil H₁;
C) optionally at least one non-volatile oil H₂, identical to or different from the oil H₁;
D) at least one colorant;
the ratio by weight of the total amount of non-volatile oil(s) to the amount of polyester being less than 8.0.

The oil H₁ and the oil H₂, which are identical or different, can be chosen from those described above.

According to a particular form, the oil H₁ and the oil H₂ are identical and denote a triglyceride of fatty acids containing from 4 to 24 carbon atoms, and more particularly a triglyceride of caprylic/capric acids (INCI name: Caprylic/Capric Triglyceride).

The oily solution of polyester of the invention can be obtained by mixing the polyester with the non-volatile oil or oils H₁ at approximately 80-100° C. The combined mixture is subsequently further cooled to 50-70° C. to be discharged from the reactor and stored.

The oily solution of polyester of the invention preferably contains the polyester at a concentration of 10% to 99% by weight, more preferentially of 30% to 90% by weight, more particularly of 50% to 80% by weight, with respect to the total weight of the mixture.

According to a preferred embodiment, the composition of the invention contains an oily solution comprising 40% by weight of triglyceride of caprylic/capric acids and 60% by weight of polyester of polyglycerol-3, of hydrogenated C₃₆ dimer acid and of isostearic acid, with respect to the total weight of the oily solution, in a molar ratio of 1/0.5/1, as described in Example 10 (copolymer) and Example 28 (oily mixture) of the document US 2021/0259945.

According to a particularly preferred form of the invention, the composition comprises an oily solution comprising:
a) a polyester obtained by reaction:
(i) of a polyglycerol-3; and
(ii) of a hydrogenated C₃₆ dimer acid; and
(iii) of isostearic acid, the components (i), (ii) and (iii) reacted being in a molar ratio of 1 mole of polyglycerol-3, of 0.5 to 1 mole of dimer acid and of 0.1 to less than 2.0 moles of fatty acids; and
b) a triglyceride of caprylic/capric acids, said mixture having, as INCI name: Diisostearoyl Polyglyceryl-3 Dimer Dilinoleate (and) Caprylic/Capric Triglyceride.

Such an oily solution is sold under the name SolAmaze Natural® by Nouryon, comprising 60% by weight, as active material, of polyester and 40% by weight of a triglyceride of caprylic/capric acids, with respect to the total weight of the oily solution.

### Colorants

The composition according to the invention comprises at least one colorant.

According to a particular form of the invention, the colorant can be chosen from pulverulent colorants, fat-soluble dyes, water-soluble dyes and their mixtures.

### Pulverulent Colorants

The pulverulent colorants can be chosen from inorganic pigments, organic pigments, pearlescent agents and their mixtures.

The term “pigments” is understood to mean white or coloured, inorganic or organic, particles which are insoluble in an aqueous medium and which are intended to colour and/or opacify the resulting composition and/or deposit. These pigments can be white or coloured, and inorganic and/or organic.

According to a particular embodiment, the pigments used according to the invention are chosen from inorganic pigments.

The term “inorganic pigment” is understood to mean any pigment which corresponds to the definition of Ullmann's Encyclopedia in the chapter, “Pigments, Inorganic”. Mention may be made, among the inorganic pigments which are of use in the present invention, of zirconium oxide or cerium oxide, and also zinc oxide, iron oxide (black, yellow or red) or chromium oxide, manganese violet, ultramarine blue, chromium hydrate and ferric blue, titanium dioxide or metal powders, such as aluminium powder and copper powder. The following inorganic pigments can also be used: Ta₂O₅, Ti₃O₅, Ti₂O₃, TiO, ZrO₂ as a mixture with TiO₂, ZrO₂, Nb₂O₅, CeO₂ or ZnS.

The size of the pigment of use in the context of the present invention is generally greater than 100 nm and can range up to 10 μm, preferably from 200 nm to 5 μm and more preferentially from 300 nm to 1 μm.

According to a particular form of the invention, the pigments exhibit a size characterized by a D[50] of greater than 100 nm and which can range up to 10 μm, preferably from 200 nm to 5 μm and more preferentially from 300 nm to 1 μm.

The sizes are measured by static light scattering using a commercial particle size analyser of MasterSizer 3000® type from Malvern, which makes it possible to determine the particle size distribution of all of the particles over a wide range which can extend from 0.01 μm to 1000 μm. The data are processed on the basis of the conventional Mie scattering theory. This theory is the most suitable for size distributions ranging from the submicronic to multimicronic; it makes it possible to determine an “effective” particle diameter. This theory is described in particular in the publication by Van de Hulst, H. C., Light Scattering by Small Particles, Chapters 9 and 10, Wiley, New York, 1957.

D[50] represents the maximum size exhibited by 50% by volume of the particles.

According to a particular form of the invention, the inorganic pigment comprises a lipophilic or hydrophobic coating; the latter is preferably present in the oily phase of the composition according to the invention.

According to a particular embodiment of the invention, the pigments can be coated according to the invention with at least one compound chosen from metal soaps; N-acylamino acids or their salts; lecithin and its derivatives; isopropyl triisostearyl titanate; isostearyl sebacate; natural plant or animal waxes; polar synthetic waxes; fatty esters; phospholipids; and their mixtures.

According to a preferential embodiment, the pigments can be coated according to the invention with an N-acylamino acid or one of its salts which can comprise an acyl group having from 8 to 22 carbon atoms, such as, for example, a 2-ethylhexanoyl, caproyl, lauroyl, myristoyl, palmitoyl, stearoyl or cocoyl group.

The amino acid can, for example, be lysine, glutamic acid or alanine. The salts of these compounds can be the aluminium, magnesium, calcium, zirconium, zinc, sodium or potassium salts. Thus, according to a particularly preferred embodiment, the pigments can be coated with an N-acylamino acid derivative which can in particular be a glutamic acid derivative and/or one of its salts, and more particularly a stearoyl glutamate, such as, for example, aluminium stearoyl glutamate. Mention may be made, as examples of pigments treated with aluminium stearoyl glutamate, of titanium dioxide pigments and black, red and yellow iron oxide pigments sold under the trade name Nai® by Miyoshi Kasei.

According to a preferential embodiment, the pigments can be coated according to the invention with isopropyl triisostearyl titanate. Mention may be made, as examples of pigments treated with isopropyl titanium triisostearate (ITT), of titanium dioxide pigments and black, red and yellow iron oxide pigments sold under the trade names BWBO-12® (Iron Oxide CI77499 and Isopropyl Titanium Triisostearate), BWYO-12® (Iron Oxide CI77492 and Isopropyl Titanium Triisostearate) and BWRO-12® (Iron Oxide CI77491 and Isopropyl Titanium Triisostearate) by Kobo.

The pigments which can be used according to the invention can also be organic pigments.

The term “organic pigment” is understood to mean any pigment which corresponds to the definition of Ullmann's Encyclopedia in the chapter, “Pigments, Organic”. The organic pigment can in particular be chosen from nitroso, nitro, azo, xanthene, quinoline, anthraquinone, phthalocyanine, metal-complex type, isoindolinone, isoindoline, quinacridone, perinone, perylene, diketopyrrolopyrrole, thioindigo, dioxazine, triphenylmethane or quinophthalone compounds.

The organic pigment(s) can be chosen, for example, from carmine, carbon black, aniline black, melanin, azo yellow, quinacridone, phthalocyanine blue, sorghum red, the blue pigments codified in the Colour Index under the references CI 42090, 69800, 69825, 73000, 74100 and 74160, the yellow pigments codified in the Colour Index under the references CI 11680, 11710, 15985, 19140, 20040, 21100, 21108, 47000 and 47005, the green pigments codified in the Colour Index under the references CI 61565, 61570 and 74260, the orange pigments codified in the Colour Index under the references CI 11725, 15510, 45370 and 71105, the red pigments codified in the Colour Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as are described in Patent FR 2 679 771.

These pigments can also be in the form of composite pigments as are described in Patent EP 1 184 426. These composite pigments can be composed in particular of particles comprising an inorganic core at least partially covered with an organic pigment and at least one binder providing the fixing of the organic pigments to the core.

The pigment can also be a lake. The term “lake” is understood to mean insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Mention may be made, among the organic dyes, of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053) or D&C Blue 1 (CI 42 090).

Mention may be made, by way of example of a lake, of the product known under the name D&C Red 7 (CI 15 850:1).

Preferably, the composition according to the invention comprises at least one pulverulent colorant of inorganic pigment type, in particular chosen from metal oxides, and more particularly chosen from titanium dioxides or iron oxides, which are or are not coated, and their mixtures.

The pearlescent agents can be chosen from white pearlescent pigments, such as mica covered with titanium or with bismuth oxychloride, coloured pearlescent pigments, such as titanium oxide-coated mica with iron oxides, titanium oxide-coated mica in particular with ferric blue or chromium oxide, or titanium oxide-coated mica with an organic pigment of the abovementioned type, and also pearlescent pigments based on bismuth oxychloride.

Preferably, the pulverulent colorant(s) is (are) present, preferably, in the composition in a content ranging from 0.5% to 30% by weight, preferably from 1% to 25% by weight, more particularly from 3% to 20% by weight, with respect to the total weight of the composition.

### Water-Soluble or Fat-Soluble Colorants

A composition according to the invention can comprise at least one water-soluble or fat-soluble colorant and preferably in a proportion of at least 0.01% by weight, with respect to the total weight of the composition.

For obvious reasons, this amount is liable to vary significantly with regard to the desired intensity of the colour effect and to the colour intensity provided by the colorants under consideration, and its adjustment clearly falls within the competence of a person skilled in the art.

The additional colorants suitable for the invention can be fat-soluble.

Within the meaning of the invention, the term “fat-soluble colorant” is understood to mean any natural or synthetic, generally organic, compound which is soluble in an oily phase or solvents miscible with a fatty substance and which is capable of imparting colour.

Mention may in particular be made, as fat-soluble dyes suitable for the invention, of synthetic or natural fat-soluble dyes, such as, for example, DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto or curcumin.

The additional colorants suitable for the invention can be water-soluble.

Within the meaning of the invention, the term “water-soluble colorant” is understood to mean any natural or synthetic, generally organic, compound which is soluble in an aqueous phase or water-miscible solvents and which is capable of imparting colour.

Mention may in particular be made, as water-soluble dyes suitable for the invention, of synthetic or natural water-soluble dyes, such as, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophyllin, methylene blue, anthocyanins (enocyanin, black carrot, hibiscus or elder), caramel or riboflavin.

### Cosmetic Compositions

The present invention also relates to a cosmetic composition comprising, in a physiologically acceptable medium, a composition as defined above.

The physiologically acceptable medium is generally adapted to the nature of the support onto which the composition has to be applied, and also to the appearance under which the composition has to be packaged.

The compositions according to the invention can additionally comprise additives commonly used in care and/or make-up products, such as:
active agents, such as vitamins, for example vitamins A, E, C and B3, adenosine, hyaluronic acid and its salts;
UV screening agents;
fillers;
waxes;
hydrophilic gelling agents;
film-forming agents;
lipophilic gelling agents;
fragrances;
preservatives;
and their mixtures.

It is a matter of routine operations for a person skilled in the art to adjust the nature and the amount of the additives present in the compositions in accordance with the invention so that the cosmetic properties desired for the compositions are not thereby affected.

### Fillers

The compositions in accordance with the invention can also comprise at least one filler which makes it possible in particular to confer on them additional properties of improved stability, wear property, coverage and/or mattness.

The term “filler” should be understood as meaning colourless or white solid particles of any shape which are provided in an insoluble form and dispersed in the medium of the composition. They make it possible to confer body or firmness on the composition and/or softness and uniformity on the make-up.

The fillers can be inorganic or organic.

Preferably, they can be chosen from natural fillers or fillers of natural origin.

The term “natural compound” is understood to mean a compound which is obtained directly from the earth or the soil, or from plants or animals, via, if appropriate, one or more physical processes, such as, for example, milling, refining, distillation, purification or filtration.

The term “compound of natural origin” is understood to mean a natural compound which has undergone one or more additional chemical or industrial treatments, bringing about modifications which do not affect the essential qualities of this compound, and/or a compound predominantly comprising natural constituents which have or have not undergone transformations. Mention may be made, as non-limiting example of additional chemical or industrial treatment bringing about modifications which do not affect the essential qualities of a natural compound, of those permitted by the controlling bodies, such as Ecocert (Reference system for biological and ecological cosmetic products, January 2003), or defined in recognized handbooks in the field, such as “Cosmetics and Toiletries Magazine”, 2005, Vol. 120, 9:10.

The fillers used in the compositions according to the present invention can be of lamellar, globular, spherical or fibrous forms or of any other form intermediate between these defined forms.

The fillers according to the invention may or may not be surface-coated, and in particular they may be surface-treated with amino acids or any other substance which promotes the dispersion and the compatibility of the filler in the composition.

### a) Inorganic Fillers

Mention may be made, as examples of inorganic fillers, of talcs, natural or synthetic micas, such as synthetic fluorphlogopites, silica, hydrophobic silica aerogels, hollow silica microspheres, kaolin, calcium carbonate, magnesium carbonate, hydroxyapatite, boron nitride, bismuth oxychloride, glass or ceramic microcapsules, or composites of silica and of titanium dioxide, such as the TSG® series sold by Nippon Sheet Glass.

### b) Organic Fillers

Mention may be made, as examples of organic fillers, of micronized natural waxes; metal soaps derived from organic carboxylic acids having from 8 to 22 carbon atoms, preferably from 12 to 18 carbon atoms, for example zinc, magnesium or lithium stearate, zinc laurate or magnesium myristate; lauroyl lysine; or cellulose powders, such as that sold by Daito in the Cellulobeads® range.

Preferably, the filler(s) are present in the composition in a content ranging from 0.5% to 20% by weight, preferably from 1% to 15% by weight, more particularly from 3% to 10% by weight, with respect to the total weight of the composition.

### Waxes

Within the meaning of the invention, the term “waxes” is understood to mean lipophilic compounds which are solid at ambient temperature (25° C.) and at atmospheric pressure (760 mmHg), with a reversible solid/liquid change of state, having a melting point of greater than or equal to 40° C. which can range up to 120° C.

The waxes described in the document Ullmann's Encyclopedia of Industrial Chemistry 2015, Wiley-VCH Verlag Gmbh & Co. KGaA, are more particularly concerned.

Such waxes can in particular be natural but also synthetic.

The term “natural” wax is understood to denote any wax pre-existing in nature or which can be transformed, extracted or purified from natural compounds existing in nature.

Mention may in particular be made, among natural waxes, of waxes referred to as fossil waxes, including those of petroleum origin, such as ozokerite, pyropissite, microcrystalline waxes also referred to as paraffins—including crude or slack waxes, slack wax raffinates, de-oiled slack wax, soft waxes, semi-refined waxes, filtered waxes, refined waxes—and microcrystalline waxes referred to as microwaxes, including bright stock slack wax. The fossil waxes also contain lignite, also referred to as montan wax, or peat wax.

Mention may be made, as natural waxes other than fossil waxes, of vegetable waxes.

Mention may be made, as examples of vegetable waxes, of carnauba wax, candelilla wax, ouricury wax, sugarcane wax, jojoba wax, Trithrinax campestris wax, raffia wax, alfalfa wax, wax extracted from Douglas fir, sisal wax, flax wax, cotton wax, Batavia dammar wax, cereal wax, tea wax, coffee wax, rice wax, palm wax, Japan wax, their mixtures and their derivatives.

Mention may be made, as natural wax other than vegetable waxes, of beeswax.

The term “synthetic” wax is understood to denote waxes, the synthesis of which requires one or more chemical reactions carried out by a human being.

Among the synthetic waxes, semi-synthetic waxes and totally synthetic waxes can be distinguished. Synthetic waxes can be waxes obtained via a Fischer-Tropsch process, for example constituted of paraffins with a number of carbon atoms ranging from 20 to 50, or polyolefin waxes, for example homo- or copolymers of ethylene, of propene or of butene, indeed even of longer-chain α-olefins. The latter can be obtained by thermomechanical degradation of plastic polyethylene, by the Ziegler process, by high-pressure processes, or also via processes catalysed by metallocene entities. These waxes can be crystallizable, partially crystallizable or amorphous. The abovementioned synthetic waxes are generally non-polar and can be chemically treated in order to obtain polar waxes, for example by one or more of the following reactions: air oxidation, grafting, esterification, neutralization with metal soaps, amidation, direct copolymerizations or addition reactions.

Preferably, the waxes will be chosen from waxes of vegetable origin such as carnauba wax.

### Gelling Agents

Depending on the viscosity of the composition which it is desired to obtain, one or more gelling agents which are hydrophilic, that is to say soluble or dispersible in water, and/or one or more gelling agents which are lipophilic, that is to say soluble or dispersible in oils, can be incorporated in a composition of the invention.

Preferably, the hydrophilic gelling agents and/or the lipophilic gelling agents will be chosen from natural gelling agents or gelling agents of natural origin.

### Hydrophilic Gelling Agents

Mention may in particular be made, as hydrophilic gelling agents, of polysaccharide biopolymers, such as pullulan, xanthan gum, guar gum, locust bean gum, acacia gum, scleroglucans, chitin and chitosan derivatives, carrageenans, gellans, alginates, celluloses, such as cellulose gums, microcrystalline cellulose, carboxymethylcellulose, hydroxymethylcellulose and hydroxypropylcellulose, and their mixtures.

### Lipophilic Gelling Agents

Mention may be made, as lipophilic gelling agents, for example, of lipophilic clays.

According to a particularly preferred form, the composition of the invention additionally contains at least one lipophilic clay.

The term “lipophilic clay” is understood to mean any clay which is fat-soluble or fat-dispersible in the oily phase of the composition.

Clay denotes a material based on hydrated aluminosilicates and/or silicates of lamellar structure.

The clays can be natural or synthetic and they are rendered lipophilic by treatment with an alkylammonium salt, such as a C₁₀ to C₂₂ ammonium chloride, in particular stearalkonium chloride or distearyldimethylammonium chloride.

They can be chosen from bentonites, in particular bentonites, hectorites and montmorillonites, beidellites, saponites, nontronites, sepiolites, biotites, attapulgites, vermiculites and zeolites.

They are preferably chosen from hectorites and bentonites.

According to a particularly preferred form, use will be made of a lipophilic clay chosen from hydrophobically modified bentonites and hydrophobically modified hectorites, in particular modified with a C₁₀ to C₂₂ quaternary ammonium chloride, such as:
a bentonite modified with stearalkonium chloride, such as the commercial products sold under the names Claytone AF®, Garamite VT®, Tixogel® LG-M, Tixogel® MP 250, Tixogel® VZ and Tixogel® VZ-V XR by BYK Additives Inc; or the commercial products sold under the name Viscogel® B3, Viscogel® B4, Viscogel® B7, Viscogel® B8, Viscogel® ED, Viscogel® GM, Viscogel® S4 and Viscogel® SD by Bentec S.P.A;
a bentonite modified with stearalkonium chloride in the presence of at least propylene carbonate and of at least one oil, such as the commercial products Dub Velvet Gum® from Stearinerie Dubois Fils, Myglyol Gel TR from Cremer Oleo, Tixogel® CGT 6030, Tixogel® DBA 6060, Tixogel® FTN, Tixogel® FTN 1564, Tixogel® IPM, Tixogel® LAN or Tixogel® LAN 1563 from BYK Additives Inc;
a hectorite modified with distearyldimethylammonium chloride (INCI name: Disteardimonium Hectorite), such as, for example, that sold under the name of Bentone® 38VCG Rheological Additive by Elementis Specialties;
a hectorite modified with distearyldimethylammonium chloride in the presence of at least propylene carbonate or triethyl citrate and of at least one oil, such as the commercial products sold under the name Bentone® Gel DOA V, Bentone® Gel EUG V, Bentone® Gel IHD V, Bentone® Gel ISD V, BentoneR Gel MIO V, Bentone® Gel PTM V, Bentone® SS-71 V, BentoneR VS-5 PC V or Bentone® VS-5 by Elementis Specialities; the commercial products sold under the name Creagel Bentone CPS/Hectone CPS or Creagel Bentone ID/Hectone ID by Créations Couleurs; the commercial products sold under the name NS Gel DM1®, NS Gel PTIS® or NS MGel 1152® by Next Step Laboratories Stop.

The lipophilic clay(s) can be present in the composition at concentrations ranging preferably from 0.1% to 5% by weight and more preferentially from 0.1% to 1% by weight, with respect to the total weight of the composition.

Mention may also be made, as lipophilic gelling agents, of esters of dextrin and of fatty acid, in particular C₁₂ to C₂₄, preferably C₁₄ to C₁₈, fatty acid, or their mixtures.

More preferentially, the dextrin ester is an ester of dextrin and of C₁₂-C₁₈, in particular C₁₄-C₁₈, fatty acid.

According to a particularly preferred embodiment, the dextrin ester is chosen from dextrin myristate and/or dextrin palmitate.

Particularly preferably, the dextrin ester is dextrin palmitate. The latter can, for example, be chosen from those sold under the names Rheopearl TL®, Rheopearl KL® and Rheopearl® KL2 by Chiba Flour Milling.

Preferably, the dextrin ester can be present in the composition at concentrations ranging preferably from 0.1% to 5% by weight and more preferentially from 0.5% to 3% by weight, with respect to the total weight of the composition.

### Film-Forming Agents

In the present patent application, the term “film-forming agent” is understood to mean any molecule capable of forming, by itself alone or in the presence of an auxiliary film formation agent, a continuous deposit on a support, at ambient temperature and atmospheric pressure.

Mention may be made, as film-forming agent, of alkylcelluloses, in particular those, the alkyl residue of which comprises between 2 and 6 carbon atoms, in particular between 2 and 3 carbon atoms.

According to a particular embodiment, the alkylcellulose defined above, and preferably ethylcellulose, represents a content ranging from 1% to 20% by weight, with respect to the weight of the composition, the weight of ethylcellulose being expressed as dry matter.

Preferentially, the composition according to the invention can comprise from 4% to 20% by weight, expressed as alkylcellulose dry matter, more particularly from 4.5% to 15% by weight, expressed as alkylcellulose dry matter, with respect to the total weight of said composition.

The alkylcellulose is a cellulose alkyl ether comprising a chain constituted of β-anhydroglucose units linked together via acetal bonds. Each anhydroglucose unit exhibits three replaceable hydroxyl groups, it being possible for all or some of these hydroxyl groups to react according to the following reaction:
RONa+R′Cl→ROR′+NaCl, where R represents a cellulose radical and R′ represents a C₂-C₆ alkyl radical.

Advantageously, the alkylcellulose can be chosen from ethylcellulose and propylcellulose.

According to a particularly preferred embodiment, the alkylcellulose can be ethylcellulose.

It is a cellulose ethyl ether.

Total substitution of the three hydroxyl groups would result, for each anhydroglucose unit, in a degree of substitution of 3, in other words in a content of alkoxy groups of 54.88%.

The ethylcellulose polymers used in a cosmetic composition according to the invention are preferentially polymers exhibiting a degree of substitution in ethoxy groups ranging from 2.5 to 2.6 per anhydroglucose unit, in other words comprising a content of ethoxy groups ranging from 44% to 50%.

The alkylcellulose can be employed in the form of a powder, such as, for example, the products of the Ethocel Standard range from Dow Chemicals or the commercial product Aqualon EC N7® sold by Ashland.

### Form of the Composition

There are many applications of the compositions according to the invention and the applications relate to all cosmetic products comprising a colorant.

The composition of the invention can be provided in the form of a milk, cream, paste and gel. It can be anhydrous or contain an aqueous phase.

Within the meaning of the invention, the expression “anhydrous composition” denotes a composition which contains less than 5% by weight of water, preferably less than 2% by weight of water, indeed even less than 0.5% by weight of water, with respect to its total weight, and in particular a composition which is devoid of water.

The compositions containing an aqueous phase can be in the form of an oil-in-water or water-in-oil emulsion, in the form of a multiple emulsion or also of a simple dispersion of an oily phase in an aqueous phase or vice versa.

The composition according to the invention can be provided in the form of a composition which is coloured, for care of the skin, in the form of a sun protection composition. If it contains cosmetic active agents, it can then be used as base for the skin, such as the hands or the face, or for the lips (lip balms, protecting the lips from the cold and/or the sun and/or the wind).

The composition of the invention can also be provided in the form of a coloured make-up product for the skin, in particular of the face, like a foundation, an eye shadow, a concealer product, a make-up product for the body, like a semi-permanent tattoo product or make-up product for the lips, like a lipstick or a lip gloss, a make-up product for keratin fibers, such as the eyelashes and the eyebrows, like a mascara or an eyeliner.

The composition according to the invention can be manufactured by the known processes generally used in the cosmetic field.

Throughout the description, including the claims, the expression “comprising a” should be understood as being synonymous with “comprising at least one”, unless otherwise specified.

The expressions “of between . . . and . . . ” and “ranging from . . . to . . . ” should be understood as meaning limits included, unless otherwise specified.

The invention is illustrated in more detail by the examples presented below. Unless otherwise indicated, the amounts indicated are expressed as percentages by weight.

### Example 1 (Invention) and Comparative Examples 1a, 1b and 1c: Foundations

Example 1 according to the invention containing the oily solution of polyester of the invention Diisostearoyl Polyglyceryl-3 Dimer Dilinoleate (60%) (and) Caprylic/Capric Triglyceride (40%) was prepared. The total amount of Caprylic/Capric Triglyceride non-volatile oil in the composition is 15% by weight, with respect to the total weight of the composition.

Comparative Examples 1a, 1b and 1c outside the invention were prepared.

Comparative Example 1a is of identical composition with the same total amount of non-volatile oil but does not contain polyester.

Comparative Example 1b is of identical composition with the same total amount of non-volatile oil but contains, instead of the polyester of the invention, the polyester Hydrogenated Castor Oil/Sebacic Acid Copolymer (Crodabond CSA®-Croda).

Comparative Example 1c is of identical composition with the same total amount of non-volatile oil but contains, instead of the polyester of the invention, the polyester Dimer Dilinoleyl Dimer Dilinoleate (Lusplan DD-DA7®-Nippon Fine Chemical).

TABLE 1

| | Example 1 | Example 1a | Example 1b | Example 1c |
|---|---|---|---|---|
| Ingredients | (Invention) | (Comparative) | (Comparative) | (Comparative) |
| Isododecane | q.s. for 100 | q.s. for 100 | q.s. for 100 | q.s. for 100 |
| Undecane (and) Tridecane | 15 | 15 | 15 | 15 |
| (Cetiol Ultimate ® - BASF) | | | | |
| Caprylic/Capric Triglyceride | 11.67 | 15 | 15 | 15 |
| Diisostearoyl Polyglyceryl-3 | 8.33 | | | |
| Dimer Dilinoleate (60%) (and) | | | | |
| Caprylic/Capric Triglyceride | | | | |
| (40%) (SolAmaze Natural ® - | | | | |
| Nouryon) | | | | |
| Hydrogenated Castor | | | 5 | |
| Oil/Sebacic Acid Copolymer | | | | |
| (Crodabond CSA ® - Croda) | | | | |
| Dimer Dilinoleyl Dimer | | | | 5 |
| Dilinoleate (Lusplan DD- | | | | |
| DA7 ® - Nippon Fine Chemical) | | | | |
| Disteardimonium Hectorite | 3 | 3 | 3 | 3 |
| (Bentone 38 VCG Rheological | | | | |
| Additive ® - Elementis) | | | | |
| Synthetic Fluorphlogopite | 1.22 | 1.22 | 1.22 | 1.22 |
| Titanium Dioxide (and) | 8.71 | 8.71 | 8.71 | 8.71 |
| Disodium Stearoyl Glutamate | | | | |
| (and) Aluminium Hydroxide/ | | | | |
| CI 77891 (and) Disodium | | | | |
| Stearoyl Glutamate (and) | | | | |
| Aluminium Hydroxide | | | | |
| Iron Oxides (and) Disodium | 0.17 | 0.17 | 0.17 | 0.17 |
| Stearoyl Glutamate (and) | | | | |
| Aluminium Hydroxide/CI | | | | |
| 77499 (and) Disodium | | | | |
| Stearoyl Glutamate (and) | | | | |
| Aluminium Hydroxide | | | | |
| Iron Oxides (and) Disodium | 0.52 | 0.52 | 0.52 | 0.52 |
| Stearoyl Glutamate (and) | | | | |
| Aluminium Hydroxide/CI | | | | |
| 77491 (and) Disodium | | | | |
| Stearoyl Glutamate (and) | | | | |
| Aluminium Hydroxide | | | | |
| Iron Oxides (and) Disodium | 1.6 | 1.6 | 1.6 | 1.6 |
| Stearoyl Glutamate (and) | | | | |
| Aluminium Hydroxide/CI | | | | |
| 77492 (and) Disodium | | | | |
| Stearoyl Glutamate (and) | | | | |
| Aluminium Hydroxide | | | | |
| Absolute Alcohol | 12 | 12 | 12 | 12 |
| % Non-Volatile Oil/% | 3.00 | ND | 3.00 | 3.00 |
| Polyester Ratio | | | | |

### Mode of Preparation

The undecane/tridecane mixture, the polyester and the isododecane were mixed in a final beaker. The mixture was stirred under a stator-rotor at 500 revolutions/min for 5 minutes.

The modified hectorite was sprinkled in under the rotor-stator at 1500 revolutions/min and the mixture was then left stirring for 15 minutes.

The mixture of pigments and of synthetic mica was sprinkled in under the rotor-stator at 2000 rpm and then the mixture was left stirring for 15 minutes.

The temperature of the bulk material was checked. If the temperature increased, the beaker was placed in a cold water bath.

The alcohol was added at the end at ambient temperature (below 30° C.) under a rotor-stator at 1000 revolutions/min. The mixture was again left stirring for 5 min and was then packaged.

### In Vitro Tests of Wear Property and of Non-Transfer

The resistance to rubbing actions and the non-transfer of Example 1 of the invention and of Comparative Examples 1a, 1b and 1c were evaluated according to the following protocol:

A 25 μm film was produced with a film drawer on an Erichsen contrast chart. This film was dried in an oven at 37° C. for 24 h. A 2 kg weight fitted with a Wypall® fabric was placed statically on the chart for 20 s, then removed.

Observation of the colouring of the Wypall® fabric gave information on the transfer and observation of the degradation of the deposit on the contrast chart gave information on the wear property of the foundations.

The colouring of the fabric and the degradation of the deposit were graded on a scale of 0 to 5. 0 corresponds to “no trace” and no degradation of the deposit and 5 corresponds to strong colouring of the Wypall® fabric and to high degradation of the deposit.

The results obtained are shown in the table below.

TABLE 2

| | Example 1 | Example 1a | Example 1b | Example 1c |
|---|---|---|---|---|
| Observations | (Invention) | (Comparative) | (Comparative) | (Comparative) |
| Degradation of the deposit | 0 | 3 | 2 | 3.5 |
| Colouring of the fabric | 0 | 3 | 1 | 2 |

From the observations and the gradings, Example 1 of the invention showed superior wear property and non-transfer qualities to those of Comparative Examples 1a, 1b and 1c.

### Example 2 (Invention) and Comparative Examples 2a and 2b: Lipsticks

Composition 2 according to the invention, containing 10% by weight of a mixture of polyester Diisostearoyl Polyglyceryl-3 Dimer Dilinoleate (6% by weight) and of Caprylic/Capric Triglyceride (4% by weight), was prepared.

Comparative Examples 2a and 2b outside the invention were prepared: Comparative Example 2a is of identical composition except that the polyester is replaced by the same content of Bis-Diglyceryl Polyacyladipate-2.

Comparative Example 2b is of identical composition except that the polyester is replaced by the same content of Hydrogenated Castor Oil Dimer Dilinoleate.

The compositions are described in detail in the table below.

TABLE 3

| Ingredients (chemical | Invention | Compar- | Compar- |
|---|---|---|---|
| name or INCI name) | 2 | ative 2a | ative 2b |
| Isododecane | 34.15 | 34.15 | 34.15 |
| Absolute Ethanol | 34.15 | 34.15 | 34.15 |
| Ethylcellulose | 11.7 | 11.7 | 11.7 |
| (Aqualon EC N7 ® - Ashland) | | | |
| Diisostearoyl Polyglyceryl-3 | 10 | | |
| Dimer Dilinoleate (and) Caprylic/ | | | |
| Capric Triglyceride | | | |
| (SolAmaze Natural ® - Nouryon) | | | |
| Bis-Diglyceryl Polyacyladipate-2 | | 10 | |
| (Softisan 649 ® - Sasol) | | | |
| Hydrogenated Castor Oil Dimer | | | 10 |
| Dilinoleate (Risocast-DA-L ® - | | | |
| Kokyu Alcohol Kogyo) | | | |
| Red 7/CI 15850 | 10 | 10 | 10 |

### Mode of Preparation of the Compositions

A part of the isododecane, the alcohol and the polyester (SolAmaze®, Softisan® or Risocast®) were mixed in a beaker. The mixture was stirred under a Rayneri flocculator at 500 rev/min for 2 minutes. The ethylcellulose was introduced as a fine mist under the deflocculator at 500 rev/min (into the vortex) and then the mixture was left stirring for 10 minutes. The pigment, ground beforehand using a three-roll mill, was added with the remaining isododecane.

### Evaluation of the Compositions:

### a) Protocol for Evaluation of the Wear Property

Each composition was applied to an Erichsen contrast chart, using a spreader, as a deposit with a thickness of 24.5 μm, over a width of at least 6 cm, and was left to dry on a heating plate at 32° C. for 40 minutes.

Three thin strips of Wypall® fabric (Kimberley Clark) of 2 cm were deposited on the deposit without them overlapping:
the first strip is dry,
the second strip is impregnated with distilled water (0.1 ml),
the third strip is impregnated with olive oil (0.1 ml).

The film drawer weighted with a weight of 2 kg was placed on all of the thin strips, and the assembly was moved over the film.

The state of the deposit was observed. The operation was repeated for each of the compositions prepared.

### b) Grading of the Wear Property

TABLE 4

| Grade | State of the deposit |
|---|---|
| 1 | Complete or almost complete removal of the deposit on the |
| | tested area; the surface of the support appears at a great |
| | many places. |
| 2 | Partial removal of the deposit on the tested area; the |
| | surface of the support appears at certain places. |
| 3 | Slight removal of the deposit, leaving the support visible |
| | in a few places |
| 4 | No significant variation in the colour of the deposit |
| | (homogeneity, colour) |
| 5 | No variation in the appearance of the deposit (homogeneity, |
| | colour) |

### c) Results:

TABLE 5

| Invention | Comparative | Comparative |
|---|---|---|
| 1 | 1a | 1c |

Deposit on application

| | | |
|---|---|---|
| Homogeneous, | Homogeneous, a | Homogeneous, |
| intense | little less intense | intense |

Deposit after the wear property tests

| | | | |
|---|---|---|---|
| While dry | 5 | 5 | 3 |
| With the water | 5 | 5 | 3 |
| With the oil | 5 | 1 | 3 |

The above results have confirmed the superiority of the composition according to the invention. The composition has made it possible in particular to obtain a homogeneous and intense deposit, the dry strength, resistance to water and resistance to oil of which have been significantly improved.

### Example 3 (Invention) and Comparative Examples 3a, 3b and 3c: Mascaras

Example 3 according to the invention containing 5% by weight of oily solution of polyester of the invention Diisostearoyl Polyglyceryl-3 Dimer Dilinoleate (60%) (and) Caprylic/Capric Triglyceride (40%) was prepared.

Comparative Examples 3a, 3b and 3c outside the invention were prepared.

Comparative Example 3a is of identical composition but contains, instead of the oily solution of polyester of the invention, the polyester Dilinoleic Acid/Butanediol Copolymer (Viscoplast 14436H®-Biosynthis) with the same amount as starting material (5% by weight).

Comparative Example 3b is of identical composition but contains, instead of the oily solution of polyester of the invention, the polyester Hydrogenated Castor Oil/Sebacic Acid Copolymer (Crodabond CSA®-Croda) with the same amount as starting material (5% by weight).

Comparative Example 3c is of identical composition but contains, instead of the oily solution of polyester of the invention, the polyester Dimer Dilinoleyl Dimer Dilinoleate (Lusplan DD-DA7®-Nippon Fine Chemical) with the same amount as starting material (5% by weight).

TABLE 6

| | Example 3 | Example 3a | Example 3b | Example 3c |
|---|---|---|---|---|
| Ingredients | (Invention) | (Comparative) | (Comparative) | (Comparative) |
| C₉₋₁₂ Alkane | q.s. for 100 | q.s. for 100 | q.s. for 100 | q.s. for 100 |
| (Vegelight Silk ® - Biosynthis) | | | | |
| Natural Waxes | 30 | 30 | 30 | 30 |
| (rice bran wax, carnauba wax) | | | | |
| Kaolin | 2 | 2 | 2 | 2 |
| (Imercare 04K ® - Imerys) | | | | |
| Iron Oxides | 4.2 | 4.2 | 4.2 | 4.2 |
| (Unipure Triple Black LC | | | | |
| 90 ® - Sensient) | | | | |
| Diisostearoyl Polyglyceryl-3 | 5 | | | |
| Dimer Dilinoleate (60%) (and) | | | | |
| Caprylic/Capric Triglyceride (40%) | | | | |
| (SolAmaze Natural ® - Nouryon) | | | | |
| Dilinoleic Acid/Butadienediol | | 5 | | |
| Copolymer (Viscoplast | | | | |
| 14436H ® - Biosynthis) | | | | |
| Hydrogenated Castor | | | 5 | |
| Oil/Sebacic Acid Copolymer | | | | |
| (Crodabond CSA ® - Croda) | | | | |
| Dimer Dilinoleyl Dimer | | | | 5 |
| Dilinoleate (Lusplan DD- | | | | |
| DA7 ® - Nippon Fine Chemical) | | | | |
| Dextrin Palmitate | 2 | 2 | 2 | 2 |
| (Rheopearl TL2 ® - Chiba | | | | |
| Flour Milling) | | | | |
| Water | 20 | 20 | 20 | 20 |
| Pullulan | 4 | 4 | 4 | 4 |
| Polyglyceryl-3 Diisostearate | 2 | 2 | 2 | 2 |
| (Lameform TGI ® - BASF) | | | | |
| Caprylyl Glycol | 0.3 | 0.3 | 0.3 | 0.3 |
| Magnesium Sulfate | 0.7 | 0.7 | 0.7 | 0.7 |
| Sodium Dehydroacetate | 0.3 | 0.3 | 0.3 | 0.3 |
| Absolute Alcohol | 3 | 3 | 3 | 3 |
| % Non-Volatile Oil/% | 0.67 | 0 | 0 | 0 |
| Polyester Ratio | | | | |

### Preparation Protocol

The starting materials were weighed out beforehand using a balance (accuracy=0.01 g). The ingredients, except the ethanol, were introduced into a manufacturing vessel where the temperature was controlled. The set temperature was fixed at 90° C. The mixture was emulsified at 90° C. after complete melting under vigorous stirring with a rotor-stator for 15 minutes. It was then cooled to 30° C. under rotor-stator stirring. The ethanol was introduced at 30° C. under rotor-stator stirring.

### Comparative Test of Wear Property of the Mascara on the Eyelashes

In order to evaluate the wear property of the mascara, each sample was applied to a false-eyelash test specimen and left to dry for 4 hours. The test specimen was subsequently sprinkled with water and was then deposited on a support. Five to-and-fro movements with a finger were carried out in order to simulate rubbing. The intensity of the black mark and the amount of black deposit lost were evaluated.

The following gradings A, B, C and D were given for the evaluation of the formation of flakes due to crumbling:
A: no crumbling was observed;
B: slight crumbling was observed;
C: crumbling was observed;
D: significant crumbling is observed.

The following gradings A, B, C and D were given for the evaluation of the tendency to form marks:
A: no mark was observed;
B: a slight mark was observed;
C: a mark was observed.

The results obtained are shown in the table below.

TABLE 6

| | Example 3 | Example 3a | Example 3b | Example 3c |
|---|---|---|---|---|
| Observations | (Invention) | (Comparative) | (Comparative) | (Comparative) |
| Wear property (staining) | A | B | C | B |
| Wear property (flakes) | A | B | B | C |

From the observations and the gradings, Example 3 according to the invention showed superior qualities of resistance to water and to rubbing actions than those of Comparative Examples 3a, 3b and 3c.

1. Liquid composition for caring for and/or making up keratin materials, comprising in particular, in a physiologically acceptable medium:
A) at least one volatile solvent;
B) at least one polyester which is the reaction product of the following components (i), (ii) and (iii):
(i) at least one polyglycerol-3;
(ii) at least one dimer acid; and
(iii) at least one fatty monoacid having from 8 to 30 carbon atoms, the components (i), (ii) and (iii) reacted being in a molar ratio of 1 mole of polyglycerol-3, of 0.5 to 1 mole of dimer acid and of 0.1 to less than 2.0 moles of fatty monoacid; and
C) optionally at least one non-volatile oil; the ratio by weight of the total amount of non-volatile oil(s) to the amount of polyester being less than 8.0; and
D) at least one colorant.

2. Composition according to claim 1, where the polyester is a substantially or completely non-sequential reaction product.

3. Composition according to claim 1, where the polyester is prepared by a one-stage process which involves the introduction of all the reactants into a reaction vessel and subsequently the induction of an entirely random addition of the dimer acid and of the isostearic acid to the polyglycerol-3.

4. Composition according to claim 1, where the polyglycerol-3 is triglycerol or a mixture of polyglycerols comprising at least triglycerol, said polyglycerols corresponding to the formula (I)
in which each Gly is independently the residue of a glycerol molecule after removal of two hydroxyl groups; and n is a mean from 2 to 10.

5. Composition according to claim 1, where the polyglycerol-3 is in the form of a mixture and is composed of at least 40% by weight, or of at least 45% by weight, or of at least 50% by weight, of a combination of diglycerol and of triglycerol, with respect to the total weight of the polyglycerol-3 in the form of a mixture.

6. Composition according to claim 1, where the polyglycerol-3 is in the form of a mixture and composed of at least 20% by weight, or of at least 25% by weight, of diglycerol; at least 15% by weight, or at least 18% by weight, of triglycerol; at least 10% by weight, or at least 12% by weight, of tetraglycerol, with respect to the total weight of the polyglycerol-3 in the form of a mixture.

7. Composition according to claim 1, where the polyglycerol-3 is in the form of a mixture and comprises at least 25% by weight of diglycerol, at least 45% by weight of triglycerol and at least 10% by weight of tetraglycerol, with respect to the total weight of the polyglycerol-3 in the form of a mixture;

8. Composition according to claim 1, where the polyester is a substantially or completely non-sequential reaction product of the following components:
(i) at least one polyglycerol-3 in the form of a mixture comprising at least 25% by weight of diglycerol, at least 45% by weight of triglycerol and at least 10% by weight of tetraglycerol, in each case with respect to the total weight of the polyglycerol-3 in the form of a mixture;
(ii) at least one hydrogenated dimer acid containing at least 60% by weight of hydrogenated C₃₆ diacid and from 5% to 25% by weight of hydrogenated C₅₄ triacid, in each case with respect to the total weight of hydrogenated acid; and
iii) isostearic acid.

9. Composition according to claim 1, where the volatile solvent is chosen from:
water:
monoalcohols comprising from 2 to 8 carbon atoms;
volatile oils;
their mixtures.

10. Composition according to claim 9, where the amount of monoalcohol(s) varies from 0% to 60% by weight, preferably from 2% to 50% by weight and more preferentially still from 3% to 40% by weight, with respect to the total weight of said composition.

11. Composition according to claim 9, where the volatile oil or oils are present at contents of less than 60.0% by weight, preferably of less than 50.0% by weight and more preferentially still of less than 40.0% by weight, with respect to the total weight of said composition.

12. Composition according to claim 1, where the ratio by weight of the total amount of volatile solvent(s) to the amount of polyester is greater than 1.0.

13. Composition according to claim 1, where the composition is anhydrous and comprises a mixture of volatile hydrocarbon oil(s) and of monoalcohol; in particular, the volatile hydrocarbon oil is chosen from isododecane, a mixture of undecane and of tridecane and their mixtures and the monoalcohol is ethanol.

14. Composition according to claim 1, where the composition is aqueous and comprises a mixture of water, of volatile hydrocarbon oil(s) and of monoalcohol, in particular a mixture of water, of C9-C14 alkanes and of ethanol.

15. Composition according to claim 1, where the amount, as active material, of polyester varies from 1% to 50% by weight, more preferentially from 1.5% to 30% by weight and more preferentially still from 2% to 20% by weight, with respect to the total weight of the composition.

16. Composition according to claim 1, where the polyester is a reaction product of polyglycerol-3, of hydrogenated C36 dimer acid and of isostearic acid in a molar ratio of 1/0.5/1.

17. Composition according to claim 1, where the ratio by weight of the total amount of non-volatile oil(s) to the amount of polyester varies from 0.5 to 5.

18. Composition according to claim 1, where the non-volatile oil is chosen from non-volatile hydrocarbon oils and more preferentially from triglycerides of fatty acids containing from 4 to 24 carbon atoms, and more particularly a triglyceride of caprylic/capric acids (INCI name: Caprylic/Capric Triglyceride).

19. Composition according to claim 1, comprising:
A) at least one volatile solvent as defined in the claims; and
B) at least one oily solution comprising:
a) at least one polyester which is the reaction product of the following components (i), (ii) and (iii):
(i) at least one polyglycerol-3;
(ii) at least one dimer acid; and
(iii) at least one fatty monoacid having from 8 to 30 carbon atoms, the components (i), (ii) and (iii) reacted being in a molar ratio of 1 mole of polyglycerol-3, of 0.5 to 1 mole of dimer acid and of 0.1 to less than 2.0 moles of fatty acids; and
b) at least one non-volatile oil H1;
C) optionally at least one non-volatile oil H2, identical to or different from the oil H1;
D) at least one colorant;
the ratio by weight of the total amount of non-volatile oil(s) to the amount of polyester being less than 8.0.

20. Composition according to claim 19, where the oil H1 and the oil H2 are identical and denote a triglyceride of fatty acids containing from 4 to 24 carbon atoms, and more particularly a triglyceride of caprylic/capric acids (INCI name: Caprylic/Capric Triglyceride).

21. Composition according to claim 19, where the oily solution of polyester contains the polyester at a concentration of 10% to 99% by weight, more preferentially of 30% to 90% by weight, more particularly of 50% to 80% by weight, with respect to the total weight of the mixture.

22. Composition according to claim 19, where the oily solution comprises 40% by weight of triglyceride of caprylic/capric acids and 60% by weight of polyester of polyglycerol-3, of hydrogenated C36 dimer acid and of isostearic acid in a molar ratio of 1/0.5/1.

23. Composition according to claim 1, comprising an oily solution comprising:
a) a polyester obtained by reaction:
(i) of a polyglycerol-3; and
(ii) of a hydrogenated C36 dimer acid; and
(iii) of isostearic acid, the components (i), (ii) and (iii) reacted being in a molar ratio of 1 mole of polyglycerol-3, of 0.5 to 1 mole of dimer acid and of 0.1 to less than 2.0 moles of fatty acids; and
b) a triglyceride of caprylic/capric acids, said mixture having, as INCI name: Diisostearoyl Polyglyclyceryl-3 Dimer Dilinoleate (and) Caprylic/Capric Triglyceride.

24. Composition according to claim 1, where the colorant is chosen from pulverulent colorants, fat-soluble dyes, water-soluble dyes and their mixtures.

25. Composition according to claim 24, where the pulverulent colorants are chosen from inorganic pigments, organic pigments, pearlescent agents and their mixtures.

26. Composition according to claim 25, where the pulverulent colorant is chosen from metal oxides, more particularly from titanium dioxides or iron oxides, which are or are not coated, and their mixtures.

27. Composition according to claim 24, where the pulverulent colorant(s) is (are) present in a content ranging from 0.5% to 30% by weight, preferably from 1% to 25% by weight, more particularly from 3% to 20% by weight, with respect to the total weight of the composition.

28. Composition according to claim 1, additionally comprising at least one additive chosen from:
active agents, such as vitamins, for example vitamins A, E, C and B3, adenosine, hyaluronic acid and its salts;
UV screening agents;
fillers;
waxes;
film-forming agents;
hydrophilic gelling agents;
lipophilic gelling agents;
fragrances;
preservatives;
and their mixtures.

29. Method for coating keratin materials, more particularly for making up and/or caring for keratin materials, such as the skin, the lips, the eyelashes and the eyebrows, characterized in that it comprises at least the application to keratin materials of a composition as defined in claim 1.
